(19) United States
(12) Patent Application Publication
(10) Pub. No.: US 2006/0127441 A1
(43) Pub. Date: Jun. 15, 2006

(54) Device for stimulating the female sexual organ, packaging and dispensing member

(75) Inventor: Johannes Anthonius Marie Lomans, Oranjestad (AW)

Correspondence Address: THE WEBB LAW FIRM, P.C., 700 KOPPERS BUILDING, 436 SEVENTH AVENUE, PITTSBURGH, PA, 15219, US

(73) Assignee: CONCEPTO WORLDWIDE LTD., ROADTOWN TORTOLA (VG)

(21) Appl. No.: 10/547,948

(22) PCT Filed: Mar. 5, 2004
(86) PCT No.: PCT/NL04/00163

§ 371 Date: Sep. 6, 2005

(30) Foreign Application Priority Data
Mar. 6, 2003 (NL) 1022860

(51) Int. Cl. A61F 6/04

(52) U.S. Cl. 424/423; 128/844; 600/38

(57) ABSTRACT

The invention relates to a device for stimulating the female sexual organ, comprising: a carrier provided with fixing means, for fixing the carrier on the male sexual organ, and a preparation for stimulating the blood flow through a body part exposed to the preparation, wherein the preparation is combined with the carrier such that the preparation is accessible from the side of the carrier directed toward the female sexual organ and the female sexual organ is exposed to the preparation when the female sexual organ is penetrated. The invention also relates to a packaging and a dispensing member.

The invention relates to a device for stimulating the female sexual organ, comprising: a carrier provided with fixing means, for fixing the carrier on the male sexual organ, and a preparation for stimulating the blood flow through a body part exposed to the preparation. The invention also relates to a packaging containing a carrier provided with fixing means for fixing the carrier on the male sexual organ, and a preparation for stimulating the blood flow through a body part exposed to the preparation. Finally, the invention also relates to a dispensing member provided with a preparation for stimulating the blood flow through a body part exposed to the preparation

Diverse aids already exist for increasing the pleasure of the woman during sexual intercourse. A determined category is intended specifically for (additional) stimulation of female organs. This particularly takes place in mechanical manner. There are also means for arousing the female sexual organ by means of arranging a cream or lotion on the organ in question. A drawback hereof is that, in the circumstances wherein the use produces the desired effect, applying such a cream or lotion is usually perceived as an inconvenient action, which inhibits the use thereof.

The object of the present invention is to provide means with which the female sexual organ can be stimulated in a manner such that this requires no, or hardly any, inconvenient and/or cumbersome actions during sexual intercourse.

The invention provides for this purpose a device of the type stated in the preamble, wherein the preparation is combined with the carrier such that the preparation is accessible from the side of the carrier directed toward the female sexual organ and the female sexual organ is exposed to the preparation when the female sexual organ is penetrated. The improved blood flow through the female sexual organ will generally result in an enhanced sensitivity, with the advantage of an increased chance of orgasm, or with a greater chance of more intense orgasms. The improved blood flow will be the particular result of local vascular dilation. More particularly the carrier can be formed by a condom and/or the carrier can comprise a ring. The arranging of such a carrier is only a simple action. In particular when the carrier is formed by a condom, arranging of the carrier often does not even require an additional action; the use of condoms already takes place on a large scale. An additional advantage of the present invention is that the use of condoms will be further stimulated, which will prevent unwanted pregnancies and the spread of sexually transmitted diseases (STD). It is anticipated that the invention will contribute toward providing a further argument for the use of condoms in addition to the existing rational arguments; it results in a higher degree of pleasure. In addition to the use of a carrier, other types of carriers can also be envisaged, such as for instance a ring whereby the female sexual organ can be stimulated in mechanical manner. When such a ring is applied, a mechanical stimulation is combined with a (chemical) stimulus of the female sexual organ, thus resulting in enhancement of the sought-after effect. In an embodiment variant which is very simple to manufacture, the carrier (respectively the condom) is coated with the preparation over at least a part of the side directed toward the female sexual organ.

Although a combination of a variant of the carrier according to the invention and a preparation whereby sexual organs are influenced is already known from U.S. Pat. No. 6,007,836, this document describes that the preparation should, conversely, be situated on the side of the condom directed toward the penis. There is even a warning against the female sexual organ also coming into contact with the preparation. This document teaches the skilled person in the field precisely the opposite of what the present invention intends.

For uniform dispensing of the preparation, the carrier can be provided with a dispensing member for containing the preparation, which dispensing member can for instance comprise an absorption member and/or a preparation container provided with at least one perforation. Uniform delivery of the preparation can result in a more prolonged activity of the sought-after effect.

For re-use of the dispensing member or the carrier, the dispensing member can also be releasable from the carrier. It is thus conceivable for instance that a re-usable carrier can be repeatedly provided with a new (filled) dispensing member. This moreover provides increased potential for cleaning of the separate components.

In a preferred variant, the preparation comprises a substrate for producing, in co-action with an enzyme in a preferred variant, nitrogen oxide (NO). Nitrogen oxide provides the effect of an improved blood flow through body parts and further has no, or practically no, undesired side-effects. The substrate can herein function as NO donor from which the NO is supplied. The invention is not limited to a specific source of nitrogen oxide; diverse substances are available for this purpose. Favourable results can be obtained when the preparation comprises arginine, more particularly L-arginine. Such a preparation has been found in practice to have a powerfully activating effect on swelling bodies.

In a particular preferred variant, the preparation can be provided with one or more additives. Colouring agents, aromatic substances and lubricants can be envisaged here. Other (natural) additives are for instance: Mentha piperata (menthol), Mentha aquatica, Mentha spicata, Menthe longifolia, Mentha suaveolens, Tunera diffusa, Leonorus cardiaca, Dioscorea villosa (wild yam), Panax ginseng, Panax pseudoginseng, Panax quinquefolius, Ginko biloba, Suma root, Camellis Sinensis (green tea), Oryza sativa, Bambusoidea, and Aloe Vera. The functionality of the device can thus be increased still further, which can result in a further stimulus for use.

The invention also provides a packaging of the type stated in the preamble, wherein the carrier and the preparation are accommodated in the packaging separately of each other. It is recommended here that the packaging is formed such that by opening the packaging the carrier and the preparation unite to form a device as described above. A device can thus be obtained as described above without the separate components (carrier and preparation) coming into contact with each other before the actual use thereof. This can for instance increase the shelf-life of the components and/or enhance the effective action of the components.

The invention further provides a dispensing member of the type stated in the preamble, comprising coupling means for connecting the dispensing member to a carrier provided with fixing means for fixing the carrier on the male sexual organ. Such a dispensing member can be releasably coupled to the carrier. The separate dispensing members with which, in combination with a (for instance already known) carrier, a device as described above can be assembled, individually also form part of the present invention. For the related advantages reference is made to the description of the device.

The invention will be further elucidated on the basis of the non-limitative exemplary embodiments shown in the following figures. Herein:

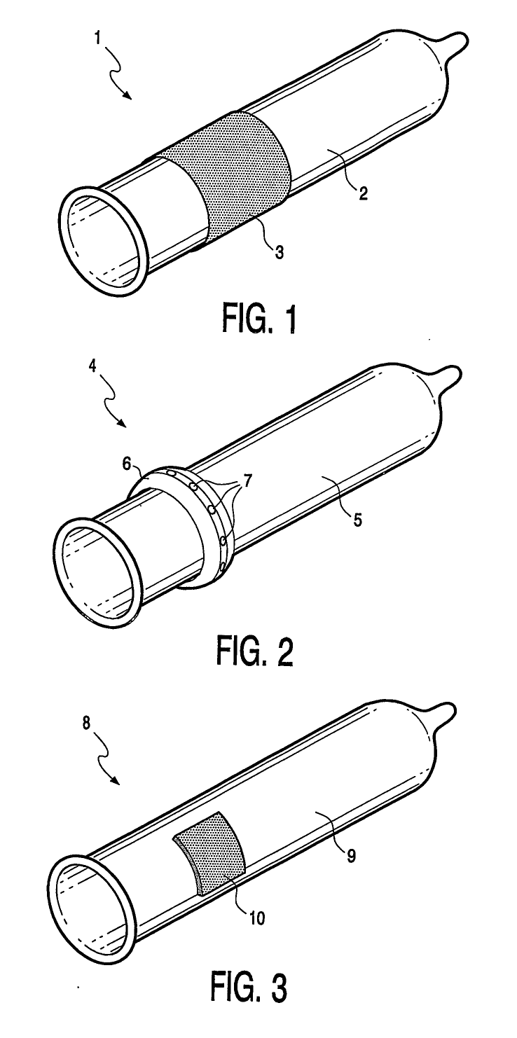

FIG. 1 shows a device according to the invention formed by a condom provided with an externally arranged preparation,

FIG. 2 shows an embodiment variant of the device according to the invention formed by a condom provided with a dispensing member in the form of a container filled with preparation,

FIG. 3 shows a subsequent embodiment variant of the device according to the invention formed by a condom provided with a dispensing member in the form of an absorption member filled with preparation,

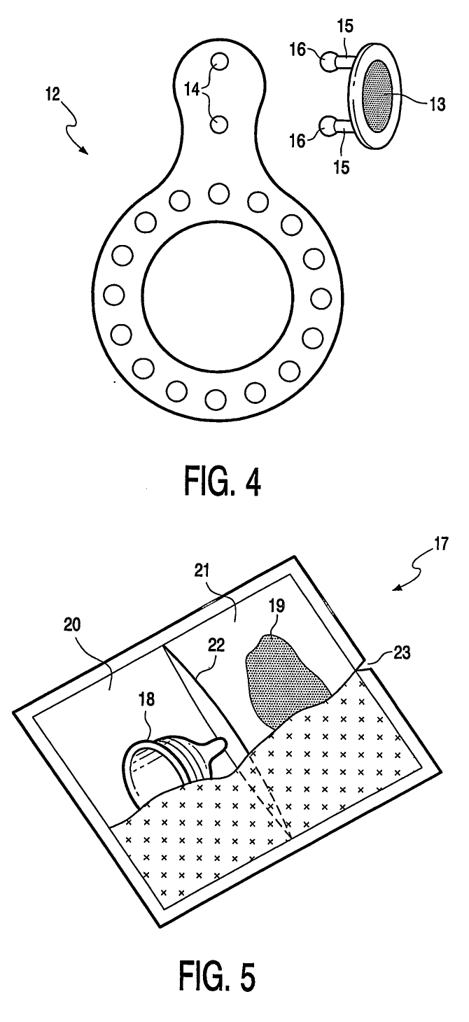

FIG. 4 shows another embodiment variant of the device according to the invention formed by a “cock ring” and a separate dispensing member which can be coupled to the “cock ring”, and

FIG. 5 shows a packaging provided with a container and a preparation for assembling a device according to the invention.

FIG. 1 shows a device 1 according to the invention in the form of a condom 2 provided with a preparation 3 arranged over a part of the outer side. Preparation 3 can be arranged as desired over the whole outer side of condom 2 or, as shown, over only a part of the outer side of condom 2. It is recommended here that the preparation is arranged precisely at the position where condom 2 comes into contact with the female organ (respectively organs) to be stimulated.

FIG. 2 shows a device 4 according to the invention in the form of a condom 5 provided with a dispensing member in the form of a container 6 filled with preparation. Container 6 is manufactured from a flexible material and provided with at least one exit opening 7. A for instance gel-like preparation, not shown, can be arranged in container 6.

In a following variant, FIG. 3 shows a device 8 according to the invention in the form of a condom 9 provided with a dispensing member filled with preparation in the form of an absorption member 10. Absorption member 10 can for instance be manufactured from a sponge-like material from which the preparation is released in delayed manner. Compare such an absorption member 10 to for instance a nicotine plaster from which nicotine is released in dosed manner for those stopping smoking.

In yet another variant, FIG. 4 shows a device 11 according to the invention in the form of a “cock ring” 12 together with a dispensing member 13 for releasable attachment to “cock ring” 12. “Cock ring” 12 can be arranged around the penis of a man and the desired effect is achieved by coupling dispensing member 13 to ring 12. Arranged in the ring to couple ring 12 and dispensing member 13 are openings 14 which can co-act with projecting parts 15 of dispensing member 13. For a reliable coupling of dispensing member 13 to ring 12, the projecting parts 15 of dispensing member 13 are provided with thickened ends 16. It is noted that “cock ring” 12 and dispensing member 13 can also be given an integrated embodiment, wherein “cock ring” 12 and dispensing member 13 are permanently connected to each other, and thus form one whole.

FIG. 5 shows a packaging 17 in a partly cut-away state, provided with a container 18 and a preparation 19 for assembling a device according to the invention. Packaging 17 is provided with two compartments 20, 21 which are mutually separated by means of a partition 22 such that container 18 (in this case a condom) and preparation 19 are completely separated from each other in the packaged state. By opening packaging 17 at the tear initiation 23, container 18 and preparation 19 will be brought into mutual contact. In another variant it is possible to envisage that container 18 can only be removed from the packaging through the compartment 21 in which preparation 19 is situated, so as to thus ensure a good contact between container 18 and preparation 19.

1-15. (canceled)

16. A device for stimulating the female sexual organ, comprising:
a carrier provided with fixing means, for fixing the carrier on the male sexual organ, and
a chemical preparation which is combined with the carrier such that the preparation is accessible from the side of the carrier directed toward the female sexual organ, and the female sexual organ is exposed to the preparation when the female sexual organ is penetrated,
wherein the preparation is adapted for stimulating the blood flow through a body part exposed to the preparation.

17. The device as claimed in claim 16, wherein the carrier is formed by a condom.

18. The device as claimed in claim 16, wherein the carrier is coated with the preparation over at least a part of the side directed toward the female sexual organ.

19. The device as claimed in claim 16, wherein the carrier comprises a ring.

20. The device as claimed in claim 16, wherein the carrier is provided with a dispensing member for containing the preparation.

21. The device as claimed in claim 20, wherein the dispensing member comprises an absorption member.

22. The device as claimed in claim 20, wherein the dispensing member comprises a preparation container provided with at least one perforation.

23. The device as claimed in claim 20, wherein the dispensing member is releasable from the carrier.

24. The device as claimed in claim 16, wherein the preparation comprises a donor of nitrogen oxide (NO).

25. The device as claimed in claim 16, wherein the preparation comprises arginine, more particular L-arginine.

26. The device as claimed in claim 16, wherein the preparation is provided with an additive.

27. A packaging, containing a carrier provided with fixing means for fixing the carrier on the male sexual organ, and a preparation for stimulating the blood flow through a body part exposed to the preparation, wherein the carrier and the preparation are accommodated in the packaging separately of each other.

28. The packaging as claimed in claim 27, wherein the packaging is formed such that by opening the packaging the carrier and the preparation are united to form a device as claimed in claim 16.

29. A dispensing member, provided with a preparation for stimulating the blood flow through a body part exposed to the preparation, comprising coupling means for connecting the dispensing member to a carrier provided with fixing means for the purpose of fixing the carrier to the male sexual organ.

30. The dispensing member as claimed in claim 29, wherein the dispensing member can be releasably coupled to the carrier.
